(19) United States
(12) Patent Application Publication
(10) Pub. No.: US 2023/0381485 A1
(43) Pub. Date: Nov. 30, 2023

(54) A Connector to Fluidically Connect a Medical Device With Air and Water Tubes

(71) Applicant: GA Health Medical Devices Limited, Mullingar, County Westmeath (IE)

(72) Inventor: Steven Chau, Hong Kong (CN)

(21) Appl. No.: 18/249,691

(22) PCT Filed: Oct. 20, 2021
(86) PCT No.: PCT/EP2021/079142

§ 371 Date: Apr. 19, 2023

(30) Foreign Application Priority Data
Oct. 20, 2020 (EP) 20202925.2

(51) Int. Cl. A61M 39/10

(52) U.S. Cl. CPC A61M 39/105

(57) ABSTRACT

A connector (20, 60) to fluidically connect a medical instrument (1) with an air tube and (34) a water tube (35), comprises a housing (21 having a first through lumen (22) with a proximal opening (26) configured to couple with the air tube (34) and a distal opening (27) configured to couple with an air port (3) of the medical instrument, and a second through lumen (25) with a proximal opening (23) configured to couple with a water tube (35) and a distal opening (24) configured to couple with a water port (2) of the medical instrument. The connector is a two-part connector comprising a proximal part (28) comprising the proximal openings (26, 23) and a distal part (29) comprising the distal openings (24, 27), wherein the proximal part and distal part are configured for detachable engagement, and wherein the second through lumen (25) comprises a non-return valve (10) disposed in the distal part of the housing. The distal openings (24, 27) are configured to fluidically connect with an air-water (AW) port (3, 2) of an endoscope. The proximal part (28) may comprise a housing section (61) dimensioned to receive the distal part (29).

### FIELD OF THE INVENTION

The present invention relates to a connector to fluidically connect a medical device with air and water tubes. In particular, the invention relates to a connector to fluidically connect an endoscope with air and water tubing leading to a water bottle

### BACKGROUND TO THE INVENTION

Endoscopes are imaging devices used to image the inside of the body. They generally have a control handle coupled to a long elongated flexible tubular body with an imaging lens and light at a distal end. Optical fibres are often employed to relay light from an illumination source in the handle to the distal tip of the elongated body. Most endoscopes incorporate air/water channels to deliver air and water to the distal tip for the purpose of cleaning the lens (water) or insufflating tissue close to the tip. The air is generally provided by a pump that is connected to an air line in the handle of the endoscope. Water is provided by an external water bottle configured for connection to the handle of the endoscope by a water line and pump. Irrigation and rinsing tube sets and systems are described in AU2011316038, U.S. Pat. No. 5,297,537 and US20180206707.

In some endoscopes, air can be routed by means of a valve to an external water bottle to pressurise the bottle. In this type of endoscope, the handle has adjacent air and water ports provided on the handle to fluidically connect a water bottle with the endoscope by means of air and water tubes. In use, air is pumped from the endoscope to the water bottle via an air tube to pressurise the bottle, and the pressure forces water in the bottle into the endoscope through the water line, where the water is used to clean the lens of the endoscope. FIGS. 1 and 2 illustrate this type of system, with a handle of an endoscope 1 with protruding water port 2 and air port 3, water bottle 4, air and water tubes 5, 6, bottle cap 7 glued to a distal end of the air and water tubes, and a connector 8 glued to a distal end of the air and water tubes and having a distal end 9 dimensioned to mate the fluidically connect with the water port 2 and air port 3. FIG. 2 shows the connector 8, tubes 5, 6 and bottle cap 7 prior to assembly.

The water bottle, tubes and connector are generally used for a 20-hour period before being disposed of. This will mean that they are used on several different patients during this period. While none of these components come into contact with the patient, there is a risk of back-flow (reflux) of water from the endoscope into the water bottle during use. This can cause contamination of water in the water bottle, which can lead to cross-contamination when the endoscope is used on a different patient using the same water bottle and tubing. This problem can be addressed by changing the whole water supply assembly after every patient, although this is an expensive solution as well as being environmentally unfriendly.

To address this problem, it is recommended to incorporate a non-return valve into the fluidic conduits to prevent reflux of water into the bottle. Incorporating a non-return valve into existing machines is expensive and time consuming. An alternative solution is to incorporate a non-return valve into the water line as illustrated in FIG. 3 in which the non-return valve 10 is provided in the water tube 6. However, while this will prevent reflux water into the bottle allowing the bottle to be re-used, the rest of the water supply assembly (cap, tubing, and connector) all must be disposed of because they are glued together and cannot be separated from the potentially contaminated connector. Again, this is an expensive solution.

It is an object of the invention to overcome at least one of the above-referenced problems.

### SUMMARY OF THE INVENTION

The Applicant has addressed the problems of the prior art by providing a modified connector, that is provided in a two-part form with a distal part detachably attached to a proximal part and where the water lumen of the distal part incorporates a one-way valve. This is illustrated in an exploded view in FIG. 5 showing the detachable distal and proximal parts and the non-return valve in the distal part. With this connector, after the endoscope has been used with a patient, the connector is detached from the air and water ports of the endoscope, and the distal part of the connector is detached from the proximal part, and a replacement distal part is attached to the proximal part to re-assemble the connector, which is then re-attached to the ports of the endoscope. In this way, only the distal part of the connector must be discarded and the remaining parts (proximal part of the connector, attached water and air tubes, bottle cap and bottle) may be re-used for the remainder of the recommended 20-hour period. This allows a kit to be supplied comprising a water bottle, a tubing assembly with bottle cap, air and water tubes, and a connector, and several replacement distal parts for the connector, that can be used with several patients during a 20-hour period without any risk of cross-contamination.

In a first aspect, the invention provides a connector to fluidically connect a medical instrument with an air tube and a water tube, the connector comprising a housing having:
a first through lumen with a proximal opening configured to couple with an air tube and a distal opening configured to couple with an air port of the medical instrument; and
a second through lumen with a proximal opening configured to couple with a water tube and a distal opening configured to couple with a water port of the medical instrument;
   characterised in that the connector is a two-part connector comprising a proximal part comprising the proximal openings and a distal part comprising the distal openings, wherein the proximal part and distal part are configured for detachable engagement, and wherein the second through lumen comprises a non-return valve disposed in the distal part of the housing.

In any embodiment, the distal openings are configured to fluidically connect with air and water ports of a scoping device. Typically, the ports are protruding ports.

In any embodiment, the distal openings are configured to fluidically connect with an air-water (AW) port of an endoscope.

In any embodiment, each proximal opening comprises a recessed socket dimensioned to receive an end of an air or water tube.

In any embodiment, the recessed socket of the first through lumen has a diameter greater than the recessed socket of the second through lumen.

In any embodiment, the proximal part and distal part are configured for detachable friction-fit engagement.

In any embodiment, a proximal end of the distal part of the housing comprises projecting ports, and a distal end of the proximal part of the housing comprises recessed ports configured to receive the projecting ports to fluidically couple the distal and proximal parts of the housing.

In any embodiment, the proximal part comprises a housing section dimensioned to receive the distal part. An example of this embodiment is illustrated in FIGS. 9 to 18.

In any embodiment, the housing section of the proximal part comprises a base section and a lid section hingedly mounted to the base section and adjustable from an open configuration to allow insertion of the distal part into the housing section and a closed configuration in which the distal part is enclosed within the housing section. The base section may be dimensioned to allow all, substantially all, or part of the distal part of the housing nest within the base when the lid is open.

In any embodiment, the lid section comprises a clamp configured to clamp the proximal part of the housing to the air port or water port of the medical instrument when the lid section is in the closed configuration. The clamp may be a C-shaped element configured to snap fit to the air or water port.

In any embodiment, the lid section comprises a clamp configured to clamp the proximal part of the housing to the air port of the medical instrument when the lid section is in the closed configuration.

In any embodiment, the distal opening of the second through lumen is recessed proximally relative to the distal opening of the first through lumen.

In any embodiment, the connector consists essentially of a proximal part comprising the housing section and the lid section, a distal part, and a non-return valve. In this embodiment, the distal part and non-return valve are disposed and detached and discarded after every use, and the proximal part may be re-used and only replaced after a defined period of use, for example 24 hours.

The invention also provides an air and water tube assembly comprising:
a connector according to the invention;
a water tube having a distal end fluidically coupled to the first through lumen of the connector via the proximal opening; and
an air tube having a distal end fluidically coupled to the second through lumen of the connector via the proximal opening.

In any embodiment, the distal ends of the air and water tubes are non-detachably attached to the connector (e.g. glued).

In any embodiment, the assembly comprises a bottle cap non-detachably connected to a proximal part of the water tube and air tube to fluidically connect a bottle with the air and water tubes when the cap is attached to the bottle. The cap generally includes bores for receipt of the distal part of the water tune and air tube. Generally, the water tube is fixed to the cap such that a distal part of the tube extends beyond the cap (allowing the tube project into the bottle and into the water in the bottle when the cap is fitted to the bottle).

In any embodiment, the water tube has a bore that is smaller than a bore of the second tube.

The invention also provides a kit of parts comprising an assembly of the invention, and additionally including one or more replacement distal parts of the connector configured for detachable attachment to the proximal part of the connector.

The invention also provides a method of using a medical instrument that employs an air and water tubing assembly according to the invention, the method comprising the steps of:
connecting the distal outlets of the connector of the assembly to air and water ports of the medical instrument;
pouring water into the bottle and securing the cap to the bottle to fluidically couple the water bottle with the medical instrument via the water tube and connector;
using the medical instrument in a medical procedure (e.g. an endoscopy of other scoping procedure);
disconnecting the connector of the assembly from air and water port of the medical instrument;
detaching the distal part of the connector from the proximal part and discarding the used distal part;
attaching a replacement distal part of the connector to the proximal part to re-assemble the connector; and
attaching the re-assembled connector to the air and water ports of the medical instrument.

In any embodiment, the medical device is an endoscope of the type having adjacent air and water ports on the handle of the endoscope. In any embodiment, the endoscope is an Olympus 025 endoscope. Other endoscopes suitable for use with the connector, assembly and methods of the invention include the Fuji 088, Fuji 090 and Pentax 072/076 endoscopes.

An additional and related aspect of the invention is an adapter for a connector of an air and water tube assembly, that allows connectors of conventional air and water tube assemblies (e.g. as shown in FIGS. 1 and 2) connect with an air and water port of a medical instrument such as a scoping device (e.g. an endoscope or the like) while allowing re-use of the air and water assembly with multiple patients. The adapter comprises a housing having:
a distal end with an air port socket dimensioned to mate with the air port of the medical instrument and a water port socket dimensioned to mate with the water port of the medical instrument;
a proximal end with an air port and a water port with the same configuration as the air and water ports of the medical instrument;
an air lumen fluidically connecting the air port socket and the air port;
a water lumen fluidically connecting the water port socket and water port; and
a non-return valve disposed in the water lumen.

Typically, the air and water port sockets of the distal end of the adapter are configured to fluidically connect with air and water ports of an endoscope, typically an Olympus 025, Fuji 088, Fuji 090 or Pentax 072/076 endoscopes.

Other aspects and preferred embodiments of the invention are defined and described in the other claims set out below.

### BRIEF DESCRIPTION OF THE FIGURES

FIG. 1 (Prior art) is an illustration of a handle of an endoscope, a water bottle, and known a water and air tube assembly for fluidically connecting the water bottle and the endoscope.

FIG. 2 (Prior art) is an illustration of the water and air tube assembly of FIG. 1 in an exploded form.

FIG. 3 (Prior art) is an illustration of a handle of an endoscope, a water bottle, and a water and air tube assembly for fluidically connecting the water bottle and the endoscope, with a non-return valve in the water tube.

FIG. 4A is a sectional view of a connector of the invention showing the distal and proximal parts prior to engagement.

FIG. 4B is an illustration of the connector of FIG. 4A with the distal and proximal parts attached together.

FIG. 5 is an illustration of an air and water tube assembly of the invention with the cap attached to a water bottle and showing the connector approaching the air and water port on the handle of an endoscope.

FIG. 6 is an exploded sectional view of the water and air tube assembly of FIG. 5 in showing the two-part connector in a detached configuration.

FIG. 7 is an illustration of an air and water port of an Olympus 025 endoscope.

FIG. 8 an illustration of a modular adapter for a connector of an air and water tube assembly, that allows connectors of conventional air and water tube assemblies as shown in FIGS. 1 and 2 connect with an air and water port of a scoping device while allowing the air and water assembly with multiple patients.

FIG. 9 is an illustration of a connector according to an alternative embodiment of the invention in which the distal part of the housing is contained within a housing section of the proximal part of the housing.

FIG. 10 is an illustration of the connector of FIG. 9 showing the lid section open and the distal part of the housing nested within the housing section of the proximal part.

FIG. 11 is an exploded view of an air and water tube assembly of the invention that incorporates a connector of FIGS. 9 and 10.

FIG. 12 illustrates the connector of FIGS. 9 and 10 attached to water and air tubes with the lid section open showing the base section and a clamp on an underside of the lid section.

FIG. 13 is an exploded view of the distal part of the connector and one-way valve prior to being placed within the distal part.

FIG. 14 is an exploded view of the connector showing how the distal part is nested within the housing section of the proximal part.

FIG. 15 illustrates the connector of FIGS. 9 to 14 attached to water tubes with the lid section open and prior to attachment of air and water ports of an endoscope.

FIG. 16 illustrates the connector attached to the air and water ports of the endoscope and prior to the lid section being closed.

FIGS. 17 and 18 illustrate how the connector is clamped to air port when the lid section of the housing is closed.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

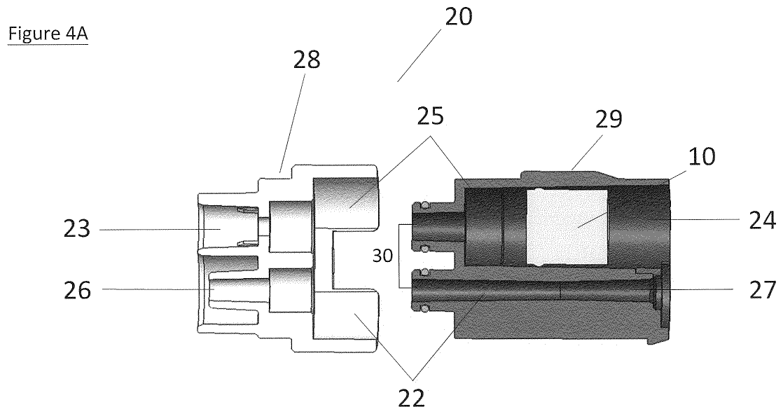

Referring to the drawings and initially to FIGS. 4A and 4B, there is illustrated a connector to fluidically connect a medical instrument with an air tube and a water tube according to the invention, indicated generally by the reference numeral 20. The connector 20 comprises a housing 21 having a first through lumen 22 for air having a proximal opening 23 and a distal opening 24, and a second through lumen for water having a proximal opening 26 and a distal opening 27. The housing 21 has a proximal part 28 comprising the proximal openings 23, 26 configured for detachable engagement with a distal part 29 comprising the distal openings 24, 27. The second through lumen 25 has a non-return valve 10 disposed in the distal part of the housing that in use prevents back-flow of water from the endoscope to the water bottle and tubing.

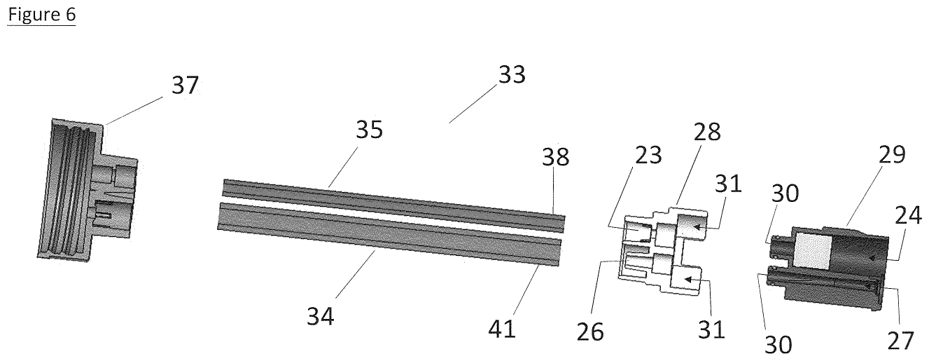

In more detail and referring to FIG. 6, a proximal end of the distal part 29 of the housing has projecting outlet ports 30 configured for friction fit engagement with corresponding sockets 31 disposed on a distal end of the proximal part 28 of the housing, to fluidically couple the distal part 29 and proximal part 28 together.

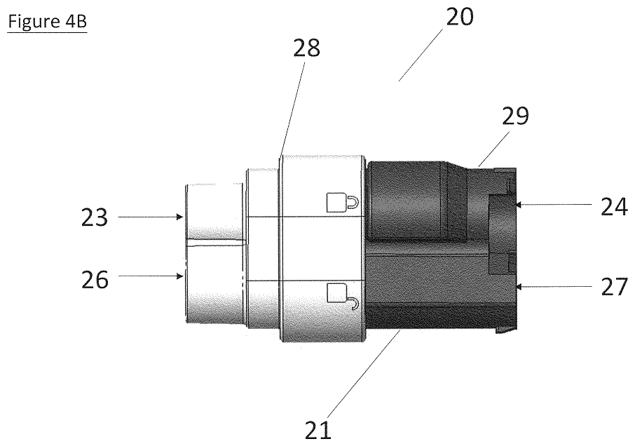

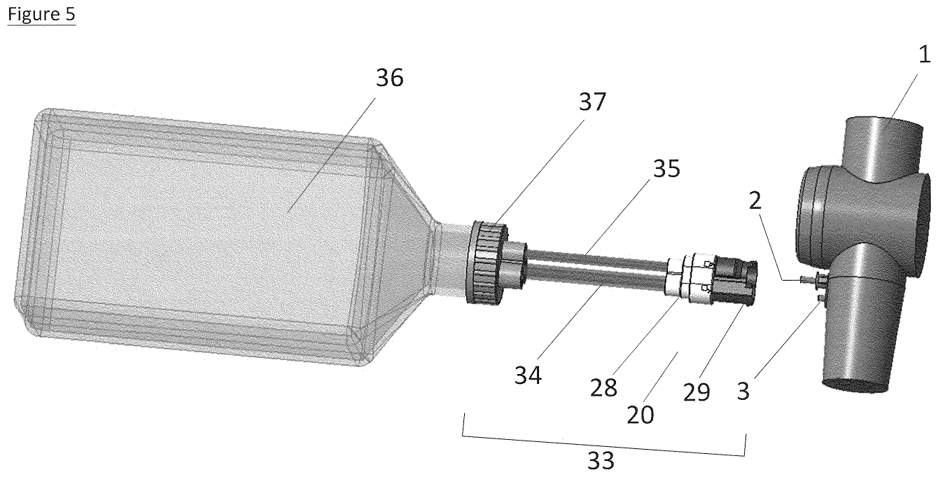

Referring to FIGS. 5 and 6, there is illustrated an air and water assembly according to the invention 33 and comprising a two-part connector 20, air tube 34, water tube 35, and bottle cap 37. The bottle cap 37 is shown attached to a water bottle 36. The air tube 33 has a distal end 38 fluidically connected to the first through lumen 21 of the connector 20 and a proximal end 39 connected to a through hole 40 in the bottle cap 37 for fluidic connection with the bottle 36. The water tube 35 has a distal end 41 fluidically connected to the second through lumen of the connector 20 and a proximal end 42 connected to a through hole 43 in the bottle cap 37 for fluidic connection with the water bottle 36 for pressurising the bottle. Although not shown, the proximal end 42 of the water tube 35 extends into the bottle to that an inlet of the water tube is under the level of the water in the bottle during use. It will be appreciated that the air and water tubes illustrated are quite short but in practice they will be much longer.

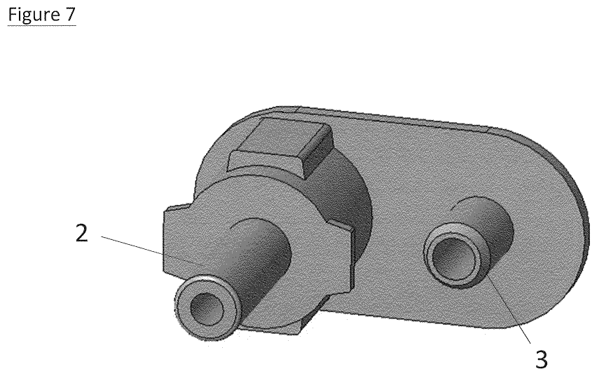

FIG. 5 also shown the handle 1 of an endoscope with air port 3 and water port 2. FIG. 7 shows the air and water ports 3, 2 in more detail.

In use, and referring to FIG. 5, the air and water assembly 33 is used to couple the air and water ports 3, 2 of the endoscope handle 1 with a water bottle 36. The bottle 36 is filled with water and the bottle cap 37 is then attached to the bottle to seal the bottle. During use of the endoscope, pressurised air is provided to the endoscope from an external pump, and upon actuation the pressurised air may be diverted to the water bottle through the connector 20 and air tube 34 to pressurise the bottle. Upon pressurisation, water in the bottle is forced into the endoscope through the water tube 35 and connector 20 where the water is routed by internal tubing a distal end of the endoscope for the purpose of cleaning the lens. The flow of water can be stopped by diverting the flow of pressurised air to the bottle through the air tube 34. During the procedure, any backflow (reflux) of water into the tubing and bottle is prevented by the non-return valve in the distal part of the connector, ensuring that the proximal part of the connector, tubing and water bottle remain sterile. After the endoscopy procedure in a patient is finished, the connector 20 is detached from the handle of the endoscope and the endoscope may then be cleaned and sterilised. To prepare the air and water assembly for the next patient, and to ensure that the tubing and bottle and water is not contaminated with biological matter from the first patient, the distal part 29 of the connector 20 (containing the non-return valve in the second through lumen) is detached from the proximal part 28 and discarded, and a replacement distal part is attached to the proximal part. The connector is then attached to the air and water ports of the sterilised endoscope, and an endoscopy procedure may be carried out on the next patient without any risk of cross-contamination. In this way, the use of the two-part connector with a non-return valve in the water lumen of the distal part allows the bottle, water, tubing, and proximal part of the connector to be re-used numerous times during a 20-hour period, with only the distal part of the connecter being discarded after every patient.

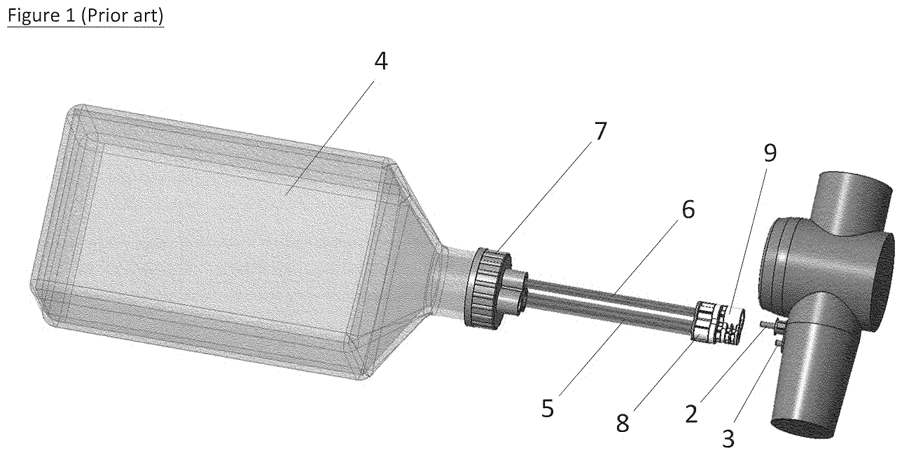

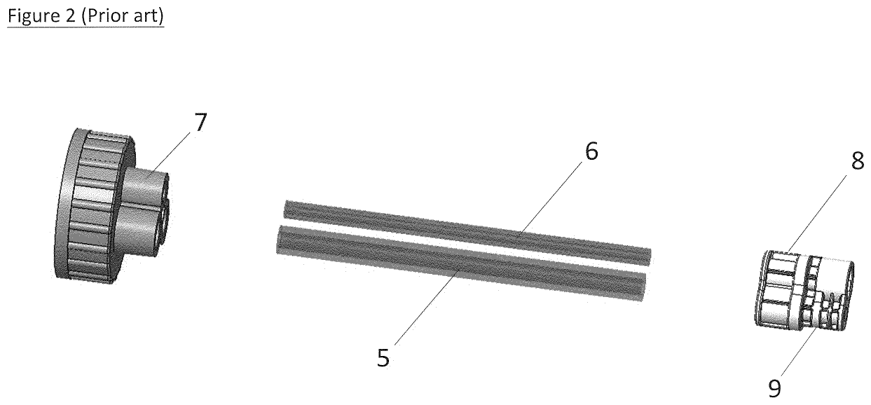

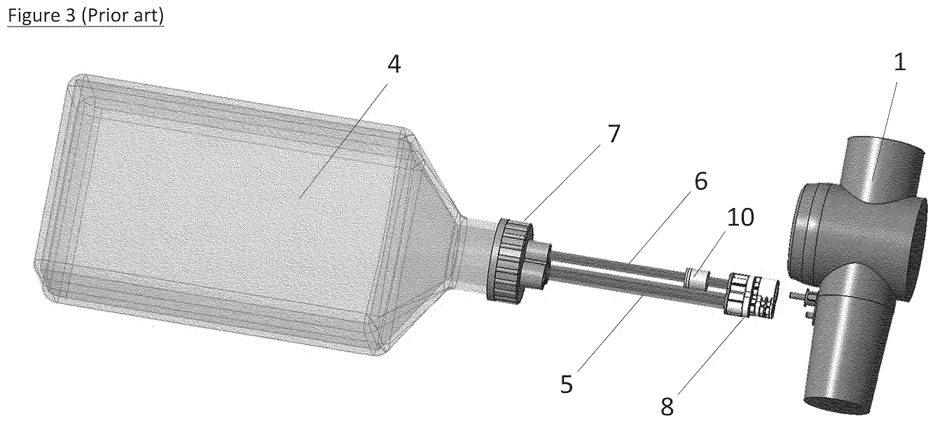

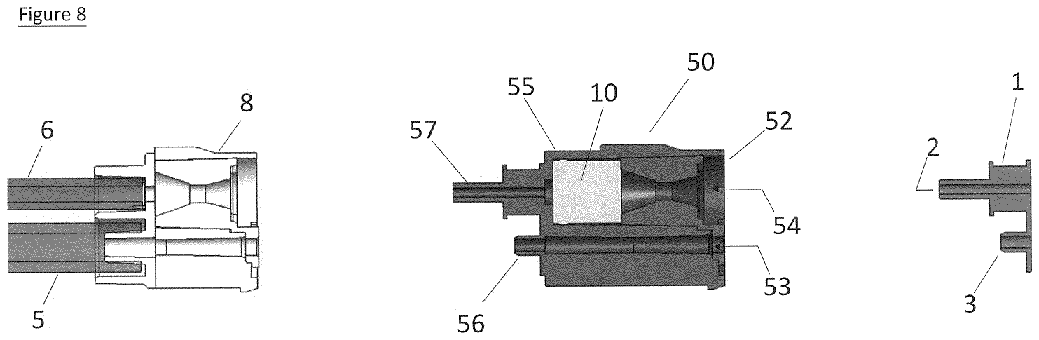

FIG. 8 illustrates an adapter fitting according to the invention, indicated generally by the reference numeral 50, in which parts described with reference to the previous embodiments are assigned the same reference numerals. The adapter fitting 50 is designed to be used with an existing air and water assembly such as that illustrated in FIGS. 1 and 2 and is used to convert a conventional one-part connector into a modified two-part connector with a non-return valve. The adapter comprises a housing 51 having a distal end 52 with an air port socket 53 dimensioned to mate with the air port 3 of an endoscope 1 and a water port socket dimensioned to mate with the water port 2 of the endoscope. A proximal end 55 comprises an air port 56 and a water port 57 identical to the air and water ports of the endoscope (in this case an Olympus 025 endoscope) and configured to fluidically couple with air and water sockets of the conventional connector 8. The adapter comprises an air lumen 58 fluidically connecting the air port socket 53 and the air port 56 and a water lumen 59 fluidically connecting the water port socket 54 and water port 57. A non-return valve 10 is disposed in the water lumen 59 to prevent backflow of water through the adapter.

In use, the adapter 50 may be coupled to a connector of a conventional air and water tube assembly, and then attached to the air and water port of an endoscope. Upon completion of the endoscopy in a first patient, the adapter is detached from the connector and discarded, and a replacement adapter attached to the connector before use in an endoscopy procedure in a second patient. The adapter functions in the same way as the distal part of the two-part connector, ensuring that if any backflow of water occurs that it does not come into contact with the connector allowing the connector and downstream tubing and bottle to be re-used.

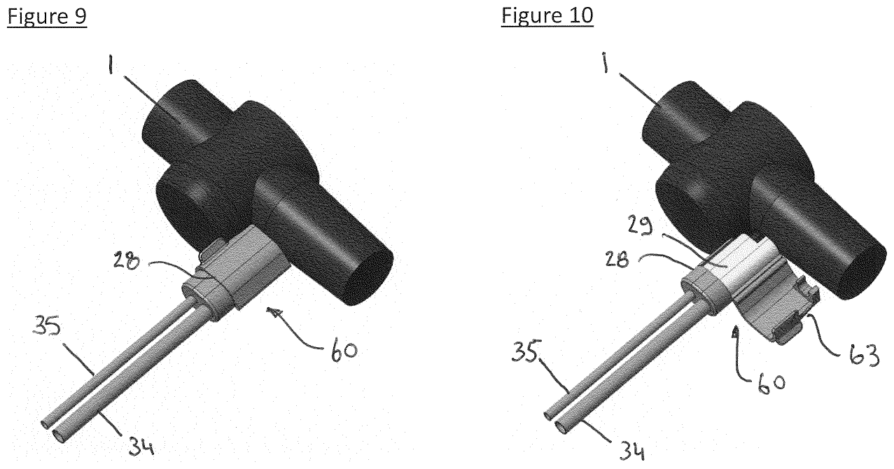

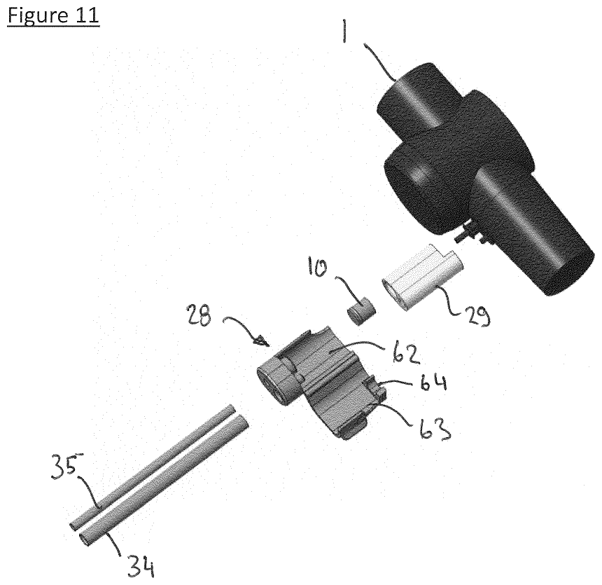

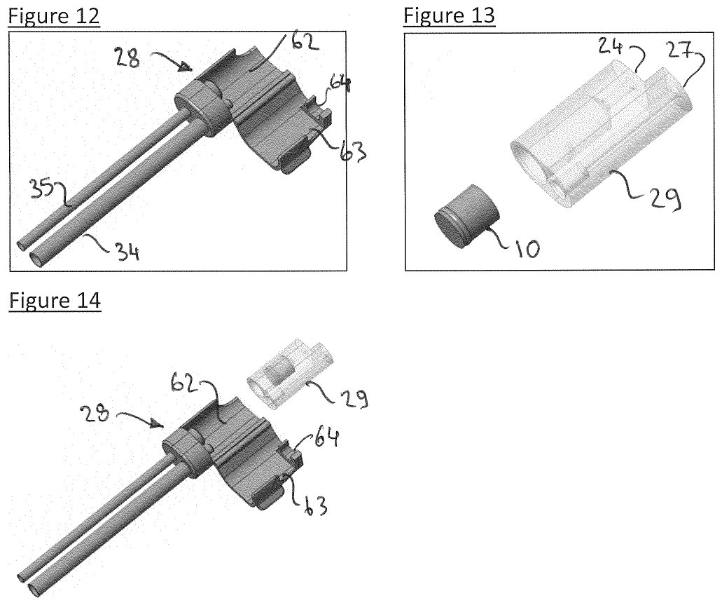

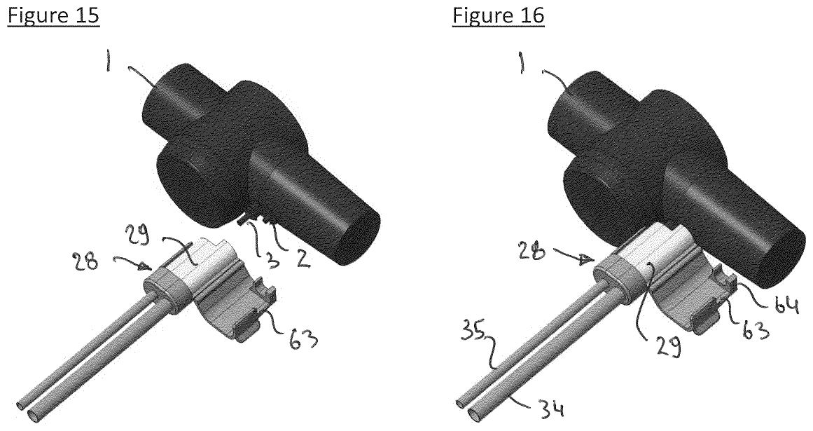

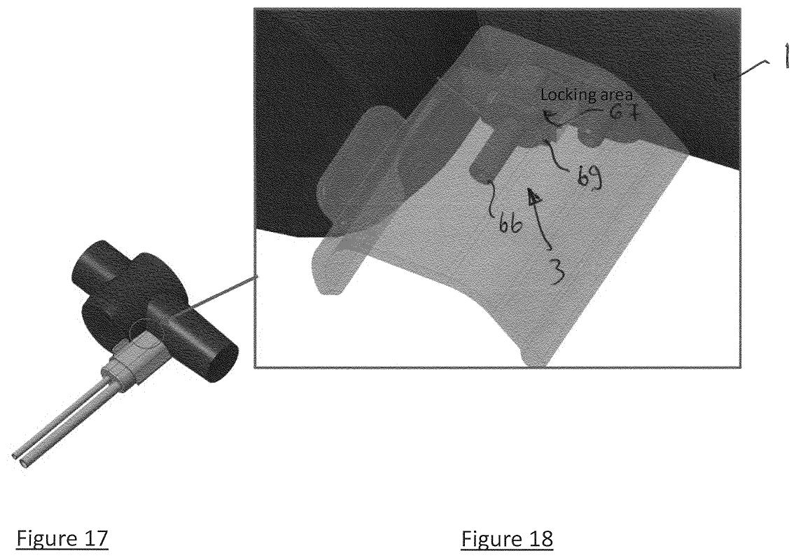

FIGS. 9 to 18 illustrate a connector according to an alternative embodiment of the invention, indicated generally by the reference numeral 60, in which parts described with reference to the previous embodiments are assigned the same reference numerals. In this embodiment, the proximal part 28 of the connector has a housing section 61 that is dimensioned to the distal part 29 of the connector is a nested arrangement. The housing section has a base section 62 and a lid section 63 hingedly mounted to the base section and adjustable from an open configuration to allow insertion of the distal part 29 into the housing section and a closed configuration in which the distal part 29 is enclosed within the housing section. The base section and lid section are dimensioned to receive and enclose the distal part of the connector when the lid section is closed, with a distal end of the housing section open to allow the distal part of the connector fluidically couple to the air and water ports of the endoscope. The distal part 29 is shaped such that the distal opening 24 of the second through lumen 25 is recessed proximally relative to the distal opening 27 of the first through lumen 22. This allows the distal opening 24 fluidically couple with a proximal end 66 the air tube 3 but also exposes a distal end of the air tube when the connector is coupled to the air tube. As illustrated in FIGS. 12 and 14, an underside of the lid section 63 incorporates a clamp 64 that is configured to snap-fit around the distal end 67 of the air tube 3 when the lid is closed. As illustrated in FIGS. 17 and 18, the annular flange 69 on the air tube 3 combined with the clamp 64 serves to lock the connector to the endoscope.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A connector (20, 60) to fluidically connect a medical instrument (1) with an air tube and (34) a water tube (35), the connector comprising a housing (21 having:
a first through lumen (22) with a proximal opening (26) configured to couple with the air tube (34) and a distal opening (27) configured to couple with an air port (3) of the medical instrument; and
a second through lumen (25) with a proximal opening (23) configured to couple with a water tube (35) and a distal opening (24) configured to couple with a water port (2) of the medical instrument;
characterised in that the connector is a two-part connector comprising a proximal part (28) comprising the proximal openings (26, 23) and a distal part (29) comprising the distal openings (24, 27), wherein the proximal part and distal part are configured for detachable engagement, and wherein the second through lumen (25) comprises a non-return valve (10) disposed in the distal part of the housing.

2. A connector (20, 60) according to claim 1, is which the distal openings (24, 27) are configured to fluidically connect with air and water ports (3, 2) of a scoping device.

3. A connector (20, 60) according to claim 1, is which the distal openings (24, 27) are configured to fluidically connect with an air-water (AW) port (3, 2) of an endoscope.

4. A connector (20, 60) according to any preceding claim, in which each proximal opening (23, 26) comprises a recessed socket dimensioned to receive an end of an air or water tube (34, 35).

5. A connector (20, 60) according to claim 4, in which the recessed socket (26) of the first through lumen has a diameter greater than the recessed socket (23) of the second through lumen.

6. A connector (20, 60) as claimed in any preceding claim, in which the proximal part (28) and distal part (29) are configured for detachable friction-fit engagement.

7. A connector (20, 60) according to any preceding claim, in which a proximal end of the distal part (29) of the housing (31) comprises projecting ports (30), and a distal end of the proximal part (28) of the housing comprises recessed ports (31) configured to receive the projecting ports to fluidically couple the distal and proximal parts of the housing.

8. A connector (60) according to any preceding claim, in which the proximal part (28) comprises a housing section (61) dimensioned to receive the distal part (29).

9. A connector (60) according to claim 8, in which the housing section (61) of the proximal part (28) comprises a base section (62) and a lid section (63) hingedly mounted to the base section and adjustable from an open configuration to allow insertion of the distal part (29) into the housing section and a closed configuration in which the distal part (29) is enclosed within the housing section.

10. A connector (60) according to claim 9, in which the lid section (63) comprises a clamp (64) configured to clamp the proximal part (28) of the housing (21) to the air port (3) or water port (2) of the medical instrument (1) when the lid section is in the closed configuration.

11. A connector (60) according to claim 9, in which the lid section (63) comprises a clamp (64) configured to clamp the proximal part (28) of the housing (21) to the air port (3) of the medical instrument (1) when the lid section is in the closed configuration.

12. A connector (60) according to claim 11, in which the distal opening (24) of the second through lumen (25) is recessed proximally relative to the distal opening (27) of the first through lumen (22).

13. An air and water tube assembly (33) comprising:
a connector (20, 60) according to any preceding claim;
a water tube (35) having a distal end fluidically coupled to the second through lumen (25) via the proximal opening (23); and
an air tube (34) having a distal end fluidically coupled to the first through lumen (25) via the proximal opening (26).

14. An assembly according to claim 13, in which the distal ends of the air and water tubes are non-detachably attached to the connector (20, 60).

15. An assembly according to claim 13 or 14, including a bottle (36) and a bottle cap (37) non-detachably connected to a proximal part of the water tube (35) and air tube (34) to fluidically connect the bottle with the air and water tubes when the cap is attached to the bottle.

16. An assembly according to any of claims 13 to 15, in which the water tube (35) has a bore that is smaller than a bore of the second tube.

17. A kit of parts comprising an assembly (33) of any of claims 13 to 116, additionally including one or more replacement distal parts (29) of the connector (20) configured for detachable attachment to the proximal part (28) of the connector.

18. An adapter (50) for a connector (8) of the type that fluidically connects an air and water tube assembly with an air and water port (3, 2) of a medical instrument such as a scoping device (1), the adapter comprising a housing (51) having:
a distal end (52) with an air port socket (53) dimensioned to mate with the air port (3) of the medical instrument (1) and a water port socket (54) dimensioned to mate with the water port (2) of the medical instrument;
a proximal end (55) with an air port (56) and a water port (57) having the same configuration as the air and water ports (3, 2) of the medical instrument (1);
an air lumen fluidically connecting the air port socket (53) and the air port (56);
a water lumen fluidically connecting the water port socket (54) and water port (57); and
a non-return valve (10) disposed in the water lumen.
